(19) United States
(12) Patent Application Publication
(10) Pub. No.: US 2022/0082578 A1
(43) Pub. Date: Mar. 17, 2022

(54) METHOD OF QUANTIFYING PSILOCYBIN'S MAIN METABOLITES, PSILOCIN AND 4-HYDROXYINDOLE-3-ACETIC ACID, IN HUMAN PLASMA

(71) Applicant: Universitätsspital Basel, Basel (CH)

(72) Inventors: Matthias E LIECHTI, Oberwil (CH); Karolina Elzbieta KOLACZYNSKA, Bottmingen (CH); Urs Philipp DUTHALER, Basel (CH)

(21) Appl. No.: 17/402,508

(22) Filed: Aug. 14, 2021

Related U.S. Application Data
(60) Provisional application No. 63/077,611, filed on Sep. 12, 2020.

(51) Int. Cl. G01N 33/94

(52) U.S. Cl. CPC G01N 33/942

(57) ABSTRACT

A method of measuring and identifying metabolites of a tryptamine compound, by obtaining a sample from an individual, and measuring and identifying metabolites of the tryptamine compound in the sample by performing a LC-MS/MS analysis. A method of adjusting dosing in patients with tryptamine compound-assisted psychotherapy in therapeutic drug monitoring (TDM), by obtaining a sample from an individual, measuring and identifying metabolites of the tryptamine compounds in the sample by performing a LC-MS/MS analysis, and adjusting a dose of the tryptamine compounds based on the measured metabolites.

### GRANT INFORMATION

Research in this application was supported in part by a grant from the Swiss National Science Foundation (Grant No. 320036_185111).

### BACKGROUND OF THE INVENTION

### 1. Technical Field

The present invention relates to compositions and methods for identification and quantification of psilocin, the active metabolite of psilocybin, and 4-hydroxyindole-3-acetic acid, the main inactive metabolite of psilocybin, in human blood plasma.

### 2. Background Art

Psilocybin is a popular recreational substance found in several species of psychedelic mushrooms (Psilocybe) which cause “mind-altering” effects in humans (Hofmann et al., 1959; Nichols, 2004). Isolated in 1958 by A. Hofmann, psilocybin's psychoactive effects are predominately mediated via 5-HT_{2A} receptors (Rickli et al., 2016; Vollenweider et al., 1998). Recently, psilocybin has been repurposed and investigated for the treatment of cluster headache, obsessive compulsive disorder, anxiety and depression, and in alcohol use disorder (Bogenschutz et al., 2018; Carhart-Harris et al., 2017; Griffiths et al., 2016; Grob et al., 2011; Johnson et al., 2017; Moreno et al., 2006; Ross et al., 2016; Sewell et al., 2006). Thus, there is a great interest in using psilocybin as a medication and a great need of further clinical studies. This also creates a need for a better understanding of the pharmacokinetics of psilocybin and psilocin and methods of quantifying these substances and their metabolites in human plasma.

Psilocybin is an indole alkaloid and structurally resembles the neurotransmitter serotonin (Hasler et al., 1997; Passie et al., 2002) (FIG. 1A). Once ingested, the prodrug psilocybin is rapidly metabolized by intestinal alkaline phosphates and nonspecific esterases to psilocin (FIG. 1B), which is responsible for psilocybin's psychoactive effects (Nichols, 2004; Rickli et al., 2016).

Subjective effects of psilocybin peak after 2 hours after oral administration and last for 6 hours (Griffiths et al., 2016; Passie et al., 2002). Consistently, psilocin reaches peak concentrations of 10-40 ng/ml in plasma 1.5-2 hours after oral administration and is eliminated with a half-life of 2-3 hours (Brown et al., 2017; Hasler et al., 1997). However, this pharmacokinetic data is preliminary and needs confirmation. For example, plasma was sampled only for 6.5 hours (Hasler et al., 1997) which does not adequately reflect the entire pharmacokinetic profile of psilocybin treatments and does not allow for the precise determination of drug exposure and elimination half-life of psilocin and 4-hydroxyindole-3-acetic acid (4-HIAA). Furthermore and importantly, psilocin undergoes glucuronidation by UDP-glucuronosyltransferases (UGT) 1A9 in the liver and UGT1A10 in the small intestine to psilocin-O-glucuronide (FIG. 1C), the major metabolite of psilocin considering that 80% is excreted from body in this form (Grieshaber et al., 2001; Hasler et al., 2002; Manevski et al., 2010). The studies conducted so far to evaluate the pharmacokinetics of psilocin did not assess both unconjugated (active) psilocin and conjugated (inactive) psilocin to validly determine the pharmacokinetic parameters of both forms of psilocin. To solve this important problem, a method of determining both forms in human plasma is first needed.

Psilocin is also deaminated and oxidized by liver aldehyde dehydrogenase and monoamine oxidase to 4-hydroxytryptophol (4-HTP) (FIG. 1E) and 4-HIAA (FIG. 1D) (Hasler et al., 1997; Lindenblatt et al., 1998; Passie et al., 2002). Valid assessments of the pharmacokinetics of these substances after controlled psilocybin administration are also lacking. With a rapidly growing interest of applying psilocybin as a potential therapeutic agent for various psychiatric disorders, it is essential to expand the knowledge of its clinical pharmacology. For instance, valid pharmacokinetic (PK) data in larger populations is needed and dose finding studies investigating the PK-pharmacodynamic (PD) relationship and drug-drug interaction studies are pending and require suitable bioanalytical methods. Pharmacokinetic data is also needed to generate reference concentration values for psilocin to adjust dosing in patients treated with psilocybin or other psilocin prodrugs. For example, plasma concentrations may be measured in patients who do not show the expected acute psychoactive response to psilocybin or an insufficient therapeutic response. To this aim, a method to measure psilocin concentrations is needed to measure the concentration in plasma at a defined time point or repeatedly (Cₘₐₓ or full PK profile) and the patient's values can then be compared with reference data from a larger population to determine correct dosing and to adjust dosing within a therapeutic drug monitoring (TDM) approach for psilocybin-assisted therapy.

Until now, most methods have focused on quantification of psilocybin's metabolites for the purpose of drug screening or preliminary pharmacokinetic studies involving limited sample size. Quantification of psilocin in plasma or urine samples was achieved by processing large amounts of sample including work-intensive extraction procedures and selective analysis was warranted using lengthy chromatographic gradient programs. Over the past years, gas chromatography (GC) and HPLC methods were developed detecting psilocin by single or tandem mass spectrometry. Even though less sample volume was required, the methods made use of time-consuming liquid-liquid or solid-phase extractions and the run time was rarely below 10 minutes. Thus, these methods are impractical for the analysis of large amounts of samples.

Therefore, there remains a need for an effective method of evaluating psilocybin metabolites in plasma.

### SUMMARY OF THE INVENTION

The present invention provides for a method of measuring and identifying metabolites of a tryptamine compound, by obtaining a sample from an individual, and measuring and identifying metabolites of the tryptamine compound in the sample by performing a LC-MS/MS analysis.

The present invention also provides for a method of adjusting dosing in patients with tryptamine compound-assisted psychotherapy in therapeutic drug monitoring (TDM), by obtaining a sample from an individual, measuring and identifying metabolites of the tryptamine compounds in the sample by performing a LC-MS/MS analysis, and adjusting a dose of the tryptamine compounds based on the measured metabolites.

### DESCRIPTION OF THE DRAWINGS

Other advantages of the present invention are readily appreciated as the same becomes better understood by reference to the following detailed description when considered in connection with the accompanying drawings wherein:

FIGS. 1A-1E are chemical structures of psilocybin and metabolites, oral psilocybin (FIG. 1A) is an inactive prodrug and is rapidly hydrolysed to psilocin, the active drug (FIG. 1B), which further undergoes a glucuronidation to psilocin-O-glucuronide (FIG. 1C), or is deamined and oxidized to 4-hydroxyindol-3-yl-acetic acid (4-HIAA) (FIG. 1D) and 4-hydroxytryptophol (4-HTP) (FIG. 1E);

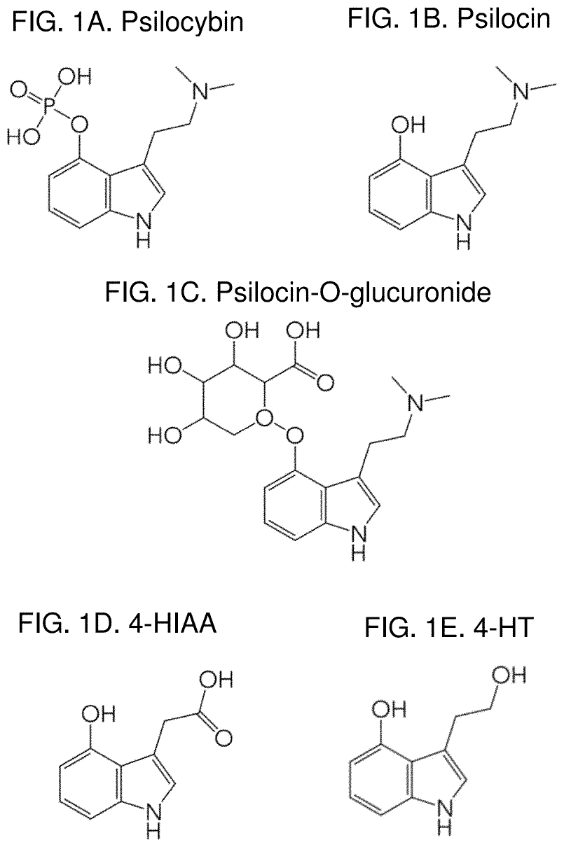

FIG. 2 is a graph of the chromatographic separation of psilocin and 4-HIAA and their respective internal standards, psilocin-d₁₀ and tryptophan-d₅, in human plasma;

FIG. 3 is a graph of 4-HIAA background noise of blank plasma recorded in positive and negative ionisation mode;

FIGS. 4A-4D are graphs of the selectivity of psilocin and 4-HIAA in blank plasma from seven different individuals, FIG. 4A shows double blank for psilocin, FIG. 4B shows blank for psilocin, FIG. 4C shows double blank for 4-HIAA, and FIG. 4D shows blank for 4-HIAA;

FIG. 5A is a table of the selectivity of psilocin and 4-HIAA in human plasma spiked to LLOQ level (0.25 ng/ml psilocin or 2.5 ng/ml 4-HIAA) compared to signals of double blank and blank, and FIG. 5B is a table of the selectivity of psilocin and 4-HIAA in human plasma spiked to LLOQ (0.25 ng/ml psilocin or 2.5 ng/ml 4-HIAA);

FIG. 6 is a table of the stability of psilocin and 4-HIAA; and

FIG. 7A is a graph of the pharmacokinetic profile of psilocin and psilocin-glucuronide, and FIG. 7B is a graph of the pharmacokinetic profile of 4-HIAA.

### DETAILED DESCRIPTION OF THE INVENTION

The present invention provides for a method of measuring metabolites of tryptamine compounds, preferably psilocybin, such as psilocin and 4-HIAA, in a human sample such as plasma. This method is validated providing information of the quality and performance of the method and an application in human subjects including a first description of the pharmacokinetics of both unconjugated and conjugated psilocin to validly derive the pharmacokinetic parameters. The invention also includes the application of the analytical method to larger phase 1 clinical studies allowing a later more comprehensive assessment of the pharmacokinetics of psilocybin.

“Sample” as used herein, refers to a sample of plasma, blood, urine, saliva, or other bodily fluid from an individual, and preferably from a human or mammal.

“Metabolite” as used herein, refers to an intermediate or end product of an original active compound as the product of metabolism. The metabolites in the present invention are preferably metabolites of psilocybin, including psilocin, 4-HIAA, psilocin-O-glucuronide, or 4-HTP. Besides, psilocybin, other prodrugs of psilocin have been described or are being developed. The method can also be used to determine psilocin and metabolites of psilocin after administration of any other prodrug of psilocin or any other psilocin analog that results in the same metabolites. Furthermore, the method can be adjusted to include the analysis of other tryptamine compounds, including analogs of psilocin, analogs of psilocybin, dimethyltryptamine (DMT), and analogs or prodrugs of DMT. This includes the analytical method as well as the concept of TDM for psilocybin-analog-assisted psychotherapy.

“LC-MSMS” as used herein, refers to a liquid chromatography-tandem mass spectrometry analytical chemistry technique.

The present invention provides for a method of measuring and identifying metabolites of psilocybin, by obtaining a sample from an individual, and measuring and identifying metabolites of psilocybin in the sample by performing a LC-MS/MS analysis. Most generally, the LC-MS/MS analysis is performed by separating analytes using a modular ultra-high performance liquid chromatography system, performing electrospray ionisation, and detecting analytes by multiple reaction monitoring.

In contrast to existing LC-MS/MS methods the present invention is further developed to be faster, using lower samples sizes, and its application also includes assessments of conjugated metabolites and the set-up of reference PK data for later TDM. This analytical method and the associated TDM application can be used to identify individuals who have taken psilocybin, and whether the psilocybin is being metabolized in the body of the individual effectively. If the metabolites are not at an expected level, dosing of the psilocybin can be adjusted in the individual as needed.

Therefore, the present invention provides for a method of adjusting dosing in patients with tryptamine compound-assisted psychotherapy in therapeutic drug monitoring (TDM), by obtaining a sample from an individual, measuring and identifying metabolites of the tryptamine compounds in the sample by performing a LC-MS/MS analysis, and adjusting the dose of the tryptamine compounds based on the measured metabolites.

A thorough development and full validation according to regulatory bioanalytical guidelines (FDA/EMA) of an LC-MS/MS method is provided for the analysis of psilocin and 4-HIAA in human plasma. Herein is a state-of-the-art LC-MS/MS method to investigate the PK of psilocybin, thereby characterizing phase I and II metabolites. The method is an improvement on other methods as it is at least 8-times more sensitive, uses small amounts of sample, involves an uncomplicated extraction protocol, and includes rapid sample analysis. In order to accomplish the aforementioned methodological advantages, samples were diluted online, enabling a semi-automated workflow to extract and analyze samples in 96-well plate format, facilitating high-throughput analysis. In addition, the method was put into practice and the clinical application of the method was demonstrated by assessing the PK of psilocin and 4-HIAA in three healthy participants in a clinical study. Furthermore, the method is used to establish reference PK data for psilocybin to support later TDM.

Psilocybin is investigated as a medication to treat a range of psychiatric disorders. Psilocin is the active metabolite of psilocybin and is a serotonergic psychedelic substance. The pharmacokinetic properties of psilocin are poorly characterized. There is a need for validly and rapidly measuring psilocin plasma levels to analyse human plasma samples from pharmacokinetics studies. 4-hydroxyindole-3-acetic acid (4-HIAA) is the main inactive metabolite of psilocybin.

Once psilocybin is marketed and regularly used in patients there is a need for TDM to determine plasma concentrations of its active metabolite psilocin. For example, plasma levels of the drug can be determined in patients not responding to usual doses of psilocybin to adjust dosing. However, a method is needed to measure psilocin concentration validly and rapidly in plasma allowing to provide physicians with such information. Additionally, psilocin to metabolite ratios can be used to identify slow or rapid metabolizers. Furthermore, metabolites with longer elimination half-lives in plasma can be used in addition to determine exposure to psilocybin and to adjust dosing. Finally, metabolite levels can be used to diagnose intoxications with psilocybin. Therefore, the present invention was developed and validated and includes a rapid LC-MS/MS method to quantify psilocin and its metabolite 4-HIAA in human plasma. Plasma samples were processed by protein precipitation using methanol. The injected sample was mixed with water in front of the C₁₈ analytical column to increase retention of the analytes. Psilocin and 4-HIAA were detected by multiple reaction monitoring in positive and negative electrospray ionisation mode, respectively.

As described in EXAMPLE 1 below, an inter-assay accuracy of 100-109% and precision of ≤8.7% was recorded over three validation runs. The recovery was near to complete (≥94.7%) and importantly, consistent over different concentration levels and plasma batches (CV %: ≤e1.1%). The plasma matrix caused negligible ion suppression and endogenous interferences could be separated from the analytes. Psilocin and 4-HIAA plasma samples can be thawed and re-frozen for three cycles, kept at room temperature for 8 hours or 1 month at −20° C. without showing degradation (≤10%). The linear range (R≥0.998) of the method covered plasma concentrations observed in humans following a common therapeutic oral dose of 25 mg psilocybin and was therefore able to assess the pharmacokinetics of psilocin and 4-HIAA. The LC-MS/MS method was convenient and reliable for measuring psilocin and 4-HIAA in plasma and is useful to facilitate the clinical development of psilocybin and TDM when psilocybin is used in patients.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

### Example 1

This example study and method description was also shown in (Kolaczynska et al., 2021). Psilocin was purchased from Lipomed (Arlesheim, Switzerland), psilocin-d₁₀ and L-ascorbic acid (AA) from Sigma-Aldrich (St. Louis, USA) and L-tryptophan-d₅ from Toronto Research Chemicals (Toronto, Canada). 4-hydroxyindole-3-acetic acid (4-HIAA) and 4-hydroxytryptophole (4-HTP) were obtained from ReseaChem (Burgdorf, Switzerland). LC-MS grade water and methanol were purchased from Merck (Darmstadt, Germany). Formic acid, and dimethyl sulfoxide (DMSO) were obtained from Sigma-Aldrich. Drug free human blood was obtained from the local blood donation center (Basel, Switzerland). Blood was collected in Lithium heparin coated S-Monovette® tubes (Sarstedt, NUmbrecht, Germany). Plasma for calibration and quality control (QC) samples was produced by centrifugation for 10 min at 4000 rpm (Eppendorf Centrifuge 5810 R).

LC-MS/MS Instrumentation and Settings

The analytes were separated using a modular ultra-high performance liquid chromatography (UHPLC) system (Shimadzu, Kyoto, Japan) consisting of four pumps (A, B, C and D). The UHPLC system was connected to a 4000 QTRAP tandem mass spectrometer (AB Sciex, Ontario, Canada).

Psilocin and 4-HIAA were retained on a Symmetry C₁₈ analytical column (3.5 μM, 100 Å, 4.6×75 mm, Waters, Mass., USA), which was heated at 45° C. in the column oven. Water was used as mobile phase A and methanol as mobile phase B. Both mobile phases contained 0.1% formic acid. The injected sample (10 μl) was mixed before the analytical column in a T-union with mobile phase A delivered by pump C. The initial flow rate of pump C was 1.3 ml/minute, which was gradually reduced during the first 0.5 minute of each run. Concurrently, pump A and B loaded the sample onto the analytical column using 10% mobile phase B and a flow rate of 0.3 mL/minute. The flow rate was steadily increased from 0.3 ml/minute to 0.5 ml/minute over the first 0.5 minute and kept at this speed until the end of the run (0.5-4.5 minutes). In order to elute the analytes, mobile B concentration was linearly increased to 95% between 0.5 and 3 minutes. Afterwards, the column was washed for 1 minute at 95% mobile B and finally re-conditioned for 0.5 minute at 10% mobile B. Between each sample injection the autosampler port was washed with a washing solution composed of water, methanol, acetonitrile, and isopropanol (1:1:1:1). The gradient program resulted in a retention time of 2.17 minutes for psilocin and psilocin-d₁₀, 2.81 minutes for tryptophan-d₅, and 3.36 minutes for 4-HIAA. Therefore, the UHPLC flow was connected with the tandem mass spectrometer only between 1.5 and 3.8 minutes of the run and otherwise directed into the solvent waste.

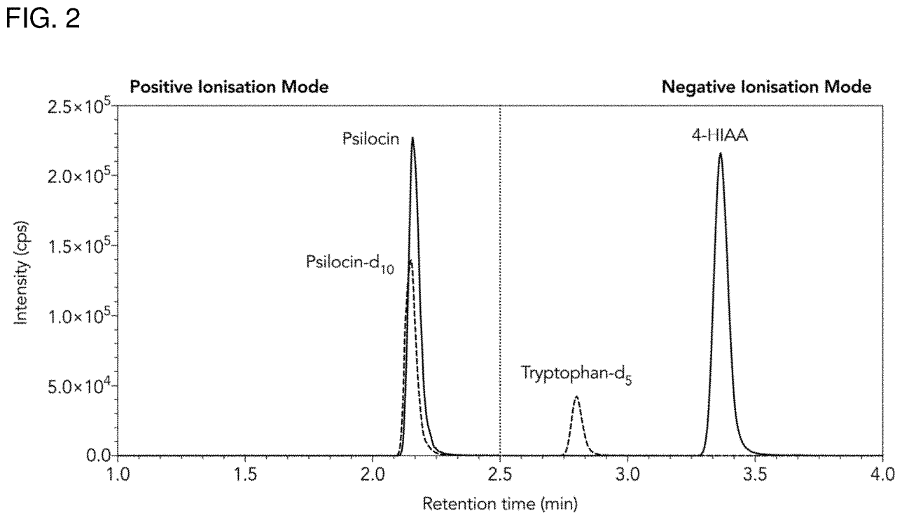

Electrospray ionisation in the positive polarity mode was used for psilocin and psilocin-d₁₀, whereas 4-HIAA and L-tryptophan-d₅ were ionised in the negative mode (TABLE 1 and FIG. 2).

TABLE 1
The detected analytes mass transitions
and mass spectrometry parameters.

| | MRM (m/z) | | | | |
|---|---|---|---|---|---|
| Analyte | (Q1→ Q3) | DP (V) | EP (V) | CE (V) | CXP (V) |
| Psilocin | 205.2→ 58.1 | 36 | 10 | 31 | 10 |
| Psilocin-d₁₀ | 215.2→ 66.0 | 36 | 10 | 25 | 12 |
| 4-HIAA | 189.9→ 130.9 | −40 | −10 | −34 | −19 |
| L-Tryp- | 208.0→ 120.0 | −80 | −10 | −26 | −17 |
| tophan-d₅ | | | | | |

MRM, Multiple reaction monitoring; m/z, mass to charge ratio; DP, Declustering potential; EP, Entrance potential; CE, Collision energy; CXP, Collision cell exit potential; V, voltage; 4-HIAA, 4-hydroxy-indole-3-acetic acid.

In FIG. 2, psilocin (100 ng/ml, black line) and psilocin-d₁₀ (10 ng/ml, dotted line) were both detected after 2.16 minutes in positive ionisation mode. The polarity mode was switched after 2.5 minutes from positive to negative electrospray ionisation in order to detect 4-HIAA (1000 ng/ml, black line) which eluted after 3.36 minutes. The retention time of tryptophan-d₅ (1000 ng/ml, dotted line), the internal standard of 4-HIAA, was at 2.81 min.

The analytes were detected by multiple reaction monitoring (MRM) by the following mass transitions (Q1→Q3): psilocin; 205.2→58.1 m/z, psilocin d-₁₀; 215.2→66.0 m/z, for 4-HIAA; 189.9→130.9 m/z, and for tryptophan-d₅; 208.0→120.0 m/z. Nitrogen was employed as ion source (gas 1; 60 l/min, gas 2; 50 l/min), curtain (10 l/min) and collision gas (4 l/min). The ion spray voltage was set at +5500 V and −4500 V in the positive and negative mode, respectively. The source temperature was 500° C.

The LC-MS/MS system was operated with Analyst software 1.7 (AB Sciex) and data were analysed with MultiQuant software 3.0.3 (AB Sciex).

Calibration and Quality Control Samples Preparation

Psilocin and 4-HIAA were weighed in duplicate in order to obtain two separate stock solutions, one for calibration samples and the other for QC sample preparations. The analytes were dissolved in DMSO containing 0.1 M ascorbic acid (DMSO-AA) to obtain a final concentration of 10 mg/ml. A calibration and QC working solution mixture of 20 μg/ml psilocin and 200 μg/ml 4-HIAA was prepared and serially diluted in DMSO-AA up to 0.025 μg/ml and 0.25 μg/ml, respectively. Calibration and QC working solutions were mixed with blank human plasma in a ratio of 1:100 (v/v). Calibration samples covered a range from 0.25-100 ng/mL for psilocin and 2.5-1000 ng/mL for 4-HIAA.

The QC samples were prepared at the lower limit of quantification (LLOQ), low concentration (QC_{LOW}), mid concentration (QC_{MID}), and high concentration (QC_{HIGH}) level corresponding to a plasma concentration of 0.25, 0.5, 10, and 50 ng/ml for psilocin and 2.5, 5.0, 100, and 500 ng/ml for 4-HIAA. All solutions were stored in light protected tubes at −20° C.

The internal standards (IS) psilocin-d₁₀ and tryptophan-d₅ were prepared in DMSO-AA at a final concentration of 10 mg/ml. An IS working solution containing 10 ng/ml psilocin-d₁₀ and 1000 ng/ml tryptophan-d₅ was made in methanol and stored at −20° C.

Sample Extraction

Aliquots of 50 μl plasma were pipetted into 96-well autosampler plates (Matrix blank storage tubes, Thermo Fischer, Massachusetts, USA) and supplemented with 5 μl of 0.1 M ascorbic acid. Next, the samples were mixed with 150 μl IS working solution and vortex mixed for 30 seconds. The extracts were then centrifuged for 30 minutes at 10° C. and 4000 rpm to receive a clear and protein free supernatant. The samples were placed inside the autosampler at 10° C., where 10 μl of the supernatant was injected into the LC-MS/MS system.

Method Validation

The analytical method was validated according to the guidelines on bioanalytical method validation of the European Medicines Agency (EMA) in terms of method linearity, accuracy and precision, selectivity and sensitivity, matrix effect and extraction recovery, and analyte stability (European Medicines Agency, 2011).

Linearity

Each calibration line consisted of two sets of a blank, a double blank, and eight calibration samples. The double blank sample was extracted with pure methanol and the other samples with IS solution. Calibration samples were analysed by increasing analyte concentration, whereas the double blank sample was injected after the upper limit of quantification (ULOQ) sample to determine the analyte carry-over between the analytical runs.

Calibration lines were established by linear regression (weighting 1/x²) of the nominal analyte concentration (x) against the analyte to IS peak area (y). Psilocin-d₁₀ was used as IS for psilocin, whereas tryptophan-d₅ was the IS of 4-HIAA. The relationship had to result in a correlation coefficient of >0.99 (R). Calibration samples with an accuracy outside of 85-115% (LLOQ: 80-120%) were excluded. However, the calibration line had to contain at least 14 determinations (>75%) including one LLOQ and one ULOQ sample.

Intra- and Inter-Assay Accuracy and Precision

The intra- and inter-assay accuracy and precision were examined by conducting three independent validation runs on three separate days. Each validation run consisted of two sets of calibration lines measured at the beginning and end of the assay. In between, seven replicates of four QC levels (LLOQ, QC_{LOW}, QC_{MID}, and QC_{HIGH}) were measured. The accuracy and precision of the method was evaluated by analysing the replicas of a single run (intra-assay, n=7) and of all three runs (inter-assay, n=21).

The precision was determined by calculating the coefficient of variation (CV %) per QC level for each individual run (intra-assay) as well as for all three runs (inter-assay). A precision of ≤15% (LLOQ: ≤20%) was acceptable.

The QC sample concentration was calculated based on the linear equation of the two calibration sets. The difference (%) between the calculated and the nominal concentration specified the accuracy of the measurement. The mean accuracy had to be between 85-115% (LLOQ: 80-120%), whereas at least 67% of all QC samples at each concentration level (intra-assay: 5 out of 7, inter-assay: 15 out of 21) had to fall within this range.

Selectivity and Sensitivity

The selectivity of the method was examined by analysing blank samples from seven different subjects. These samples were processed with and without IS to determine if interference is caused by components of the plasma matrix or the IS itself, respectively. Furthermore, each blank sample was spiked at the LLOQ concentration (psilocin: 0.25 ng/ml or 4-HIAA: 2.5 ng/ml) to evaluate the sensitivity of the method. The method was considered to be selective if the LLOQ signal intensity was at least five times higher than the background noise of blank plasma. To validate the method sensitivity, the LLOQ samples of seven different batches of plasma had to display a precision of ≤20% and a mean accuracy of 80-120%, where at least 67% of the samples had to lie within these limits.

Extraction Recovery and Matrix Effect

The extraction recovery and matrix effect were investigated for seven different plasma batches at the LLOQ, QC_{LOW}, QC_{MID}, and QC_{HIGH} concentration levels.

The extraction recovery was estimated by spiking blank plasma (before extraction) and blank plasma supernatants (after extraction) using equal amounts of analyte. The peak area found in the spiked supernatant corresponded to 100% recovery and was compared to the peak area of spiked and processed plasma samples.

The matrix effect was determined by comparing the analyte peak area in samples with and without matrix. Therefore, pure water and extracted blank plasma (after extraction) were prepared with equal amounts of analyte. The ratio (%) of the analyte peak area in plasma extracts to the peak area in water corresponded to the matrix effect.

Overall, the recovery and matrix effect had to be consistent with a CV % of less than 15% between different plasma batches and concentration levels.

Stability

The stability of psilocin and 4-HIAA in plasma were investigated under different storage conditions. Seven replicates of LLOQ, QC_{LOW}, QC_{MID}, and QC_{HIGH} samples were stored for 8 hours at room temperature (bench-top stability) and for 1 month at −20° C. (one-month stability). Moreover, the stability was assessed after three consecutive freeze and thaw cycles (freeze/thaw stability), thereby freezing the QC samples at −20° C. for at least 24 hours and thawing them afterwards at room temperature. The concentration of those stability test samples was calculated based on a freshly prepared calibration line. Samples were designated to be stable if the accuracy was between 85-115% (LLOQ: 80-120%) and the precision ≤15% (LLOQ: ≤20%).

Method Application

To examine the application of the developed method, psilocin and 4-HIAA concentrations were quantified in plasma samples of three healthy volunteers receiving a single oral dose of 25 mg psilocybin. This is a moderate to high dose currently used in clinical phase 2-3 studies. The study was approved by the Ethical Committee of Northwestern and Central Switzerland (EKNZ, BASEC ID: 2019-00223), registered at ClinicalTrials.gov (ID: NCT03912974), and conducted in accordance with the Declaration of Helsinki and International Conference on Harmonization Guidelines in Good Clinical Practice. All volunteers provided written informed consent prior to study participation.

To establish concentration time profiles, blood samples were collected in lithium heparin coated tubes at the following time points: 2 hours before and 0, 15, 30, 45, 60, 90, 120, 150, 180, 240, 300, 360, and 420 minutes after treatment. Blood samples were centrifuged for 10 minutes at 3000 rpm to obtain plasma, which was transferred into cryotubes. All samples were stored −80° C. until analysis.

Study, calibration, and QC samples were processed as described before. In addition, the total amount of glucuronide conjugated psilocin and 4-HIAA was determined similar to the protocol of Kamata et al. (2006). In brief, 5 μl of Escherichia coli deglucuronidase (3000 units/ml in water) was mixed with 50 μl plasma sample. An aliquot of 100 μl acetic acid buffer (0.1 M) was added to the mixture. The samples were incubated for 3 hours at 37° C. in a thermomixer (Eppendorf, Hamburg, Germany). Enzymatic reaction was terminated, and samples extracted by the addition of 150 μl methanol. The samples were vortex mixed and centrifuged as outlined above.

For each analytical run, a calibration line was analysed at the beginning and at the end of the measurements. In between, the study samples of the three volunteers were measured as well as triplicates of LLOQ, QC_{LOW}, QC_{MID}, and QC_{HIGH} samples. Samples with a concentration below the LLOQ were marked as blq (below limit of quantification) and samples with concentrations above the ULOQ were diluted with blank plasma into the calibration range.

The concentration-time profile was plotted and the maximal plasma concentration (Cₘₐₓ) and time to reach it (Tₘₐₓ) were obtained graphically from the plots. Pharmacokinetic parameters were calculated using non-compartmental methods in Phoenix WinNonlin 8.3 (Certara, N.J., USA). The area under the plasma concentration time profile was calculated by using the linear trapezoidal rule from 0-420 min (AUC_{LAST}). The elimination half-life (t_{1/2}) was calculated by the equation

$${t}_{1 / 2} = \frac{0.693}{λ} ,$$

where the elimination rate constant (λ) was the slope of log(C(t)) versus t determined in the terminal elimination phase.

Results of the Method Validation and Application

An LC-MS/MS method was developed and fully validated with a simple and fast sample analysis workflow.

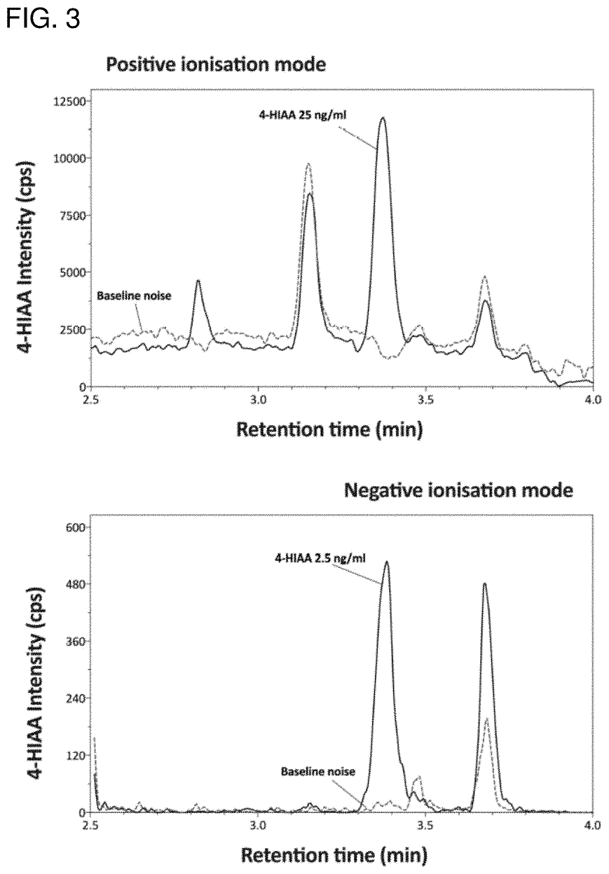

First, the ionisation and fragmentation parameters of psilocin, psilocin-d₁₀, 4-HIAA, 4-HTP, and tryptophan-d₅ were optimized by infusing the analytes into the mass spectrometer (TABLE 1). Positive and negative polarity ionisation were tested for 4-HIAA considering that it possesses an amine and carboxylic acid functional group, whereas psilocin and 4-HTP were tuned only in the positive mode. A screening of the most abundant fragments was performed to allow quantification by multiple reaction monitoring (MRM). Psilocin (205.2 m/z) broke down most abundantly to the fragments 58.1 and 160.0 m/z, while psilocin-d₁₀ fragmented into 66.0 and 164.0 m/z retaining eight and four deuterium atoms, respectively. Hence, fragment 58.1 m/z consists of the trimethylamine and 164 m/z of the 2-(Indo)-3-yl)-ethyl constituent of psilocin. Importantly, both fragments were also reported and used as quantifier ions by others (Björnstad et al., 2009; del Mar Ramirez Fernandez et al., 2007; Kamata et al., 2003; Kamata et al., 2006; Martin et al., 2012). 4-HIAA and 4-HTP were not yet detected by tandem mass spectrometry. Fragment 146 m/z and 130.9 m/z were most abundant for 4-HIAA in the positive and negative mode, respectively. 4-HTP fragmented predominantly into 160.1 m/z as observed for psilocin supporting that this fragment corresponds to the protonated 2-(Indol-3-yl)-ethyl moiety of psilocin. The validation was initially launched by ionising all analytes positively to avoid polarity switching. However, the employed mass transition of 4-HIAA (192.1→146 m/z) resulted in pronounced interferences with endogenous plasma components, which were difficult to separate from the analyte signal. In the negative mode, however, the baseline noise was negligible for 4-HIAA compared to positive mode (FIG. 3). In FIG. 3, a chromatogram of a plasma sample containing either 25 ng/ml (positive mode) or 2.5 ng/ml (negative mode) 4-HIAA was overlaid with a blank plasma chromatogram. In the positive ionisation mode, 4-HIAA was detected by the mass transition 192.1→146.0 m/z. A pronounced background noise of 2500 counts per seconds (cps) was observed in blank plasma samples, which interfered with the 4-HIAA signal. The mass transition 189.9→130.9 m/z was employed for 4-HIAA in the negative mode, resulting in a negligible background noise of <100 cps in blank plasma. Thus, polarity switching was unavoidable and tryptophan-d₅ had to be incorporated into the method as IS of 4-HIAA.

Next, the chromatography of the analytes was optimized in order to concentrate and separate them on the analytical column. A large variety of columns were screened showing that pentafluorophenyl (PFP) and biphenyl phases were able to retain the relatively polar and aromatic analytes. In addition, C₁₈ columns such as Symmetry C₁₈ column, which feature an alkyl ligand density that allows the analyte to interaction with polar siloxane and silanol surface functionalities, produced good analyte retention and symmetric peak shapes. Methanol and acetonitrile resulted in similar analyte peak intensities, whereas the acetonitrile eluted the analytes faster. Several MS compatible modifiers (formic and acetic acid, or ammonium formate, acetate and fluoride) were investigated. The addition of ammonium fluoride to both mobile phases enlarged the linear range of the method for biphenyl phase columns. However, the additive reduced also the durability of some columns (e.g., Symmetry C₁₈). 4-HIAA eluted considerably later than psilocin, though at the same time with 4-HTP making polarity switching difficult. Lastly, 4-HTP was not included in the method, because it was not detectable at a limit of detection of 2.5 ng/ml in plasma of volunteers who received 25 mg psilocybin.

Finally, different plasma protein precipitation solvents were investigated for a simple sample extraction. Methanol, acetonitrile, and ethanol yielded comparable signal intensities. However, the peak shape of the analytes was poor because the injected sample consisted of mainly organic solvent. Evaporating the extracts and resuspending the residuals in mobile phase A solved the problem. In addition, protein precipitation with perchloric acid (1 M) was evaluated because it could be expected that the hydrophilic analytes are efficiently extracted and retained on the analytical column by the aqueous solvent. Indeed, the extraction with perchloric acid was promising, however the supernatant of the extract had still to be transferred into another tube to neutralize the pH to prevent the column from damage.

Finally, plasma samples were extracted with methanol, plasma proteins were centrifuged to the bottom of the tubes, and an aliquot of the supernatant was injected into the LC-MS/MS system. Sharp and symmetric peaks were obtained by extensively mixing the injected sample with water within a T-union, which was installed in front of the analytical column. This semi-automated workflow allowed to extract and analyse the plasma samples in single tubes or 96 well plate format and facilitates the analysis of large amounts of samples.

Method Validation

Method Linearity, Accuracy, and Precision

Three validation runs were performed including four QC levels (LLOQ, QC_{LOW}, QC_{MID}, and QC_{HIGH}) with seven replicates and two calibration lines per run. In total, 54 calibration and 84 QC samples were analysed per analyte.

The method was linear over a range of 0.25 to 100 ng/ml for psilocin and 2.5 to 1000 ng/ml for 4-HIAA with a correlation coefficient of >0.998. All 4-HIAA calibrators passed the inclusion criteria, whereas only one psilocin calibrator exhibited an accuracy bias of more than 15%. The calibration range chosen for both analytes was suitable to quantify clinical samples. It encompassed concentrations that were approximately five times above the expected maximal plasma concentrations, but also low concentration samples observed during early drug absorption and in late elimination phase (Brown et al., 2017; Hasler et al., 1997; Lindenblatt et al., 1998).

The intra-assay precision of psilocin was ≤9.1% and of 4-HIAA 6.5%, while the inter-assay precision was ≤8.7% (TABLE 2). Furthermore, the mean intra-assay accuracy observed for psilocin was between 96.3-109% and for 4-HIAA between 97.5-109%, whereas the inter-assay accuracy bias was ≤9.0%. None of the psilocin QC samples were outside 85-115% accuracy (LLOQ: 80-120%) and only two out of 84 QC samples did not pass the acceptance criteria in case of 4-HIAA.

TABLE 2
The intra- and interday accuracy and precision calculated for psilocin and 4-HIAA in human plasma.

| | | CO | | CO | | CO | | CO | |
|---|---|---|---|---|---|---|---|---|---|
| | Cnominal | Assay 1 | | Assay 2 | | Assay 3 | | Assay 1-3 | |
| Analyte | (ng/ml) | (ng/ml) | Accuracy ± CV (%) | (ng/ml) | Accuracy ± CV (%) | (ng/ml) | Accuracy ± CV (%) | (ng/ml) | Accuracy ± CV (%) |
| Psilocin | 0.250 | 0.241 | 96.3 ± 7.7 | 0.249 | 100 ± 5.5 | 0.259 | 108 ± 9.1 | 0.253 | 101 ± 5.7 |
| | 0.500 | 0.502 | 100 ± 4.6 | 0.513 | 103 ± 3.5 | 0.516 | 103 ± 8.5 | 0.511 | 102 ± 5.8 |
| | 10.0 | 10.6 | 106 ± 3.4 | 10.9 | 109 ± 3.4 | 10.6 | 106 ± 3.7 | 10.7 | 107 ± 3.6 |
| | 50.0 | 51.5 | 103 ± 2.3 | 51.8 | 104 ± 2.3 | 48.9 | 97.9 ± 3.9 | 50.8 | 102 ± 3.8 |
| 4-HIAA | 2.5 | 2.58 | 107 ± 5.9 | 2.80 | 112 ± 3.7 | 2.73 | 109 ± 4.4 | 2.74 | 109 ± 4.9 |
| | 5.0 | 5.05 | 101 ± 2.8 | 5.50 | 110 ± 4.1 | 5.02 | 101 ± 6.5 | 5.19 | 104 ± 5.2 |
| | 100 | 106 | 106 ± 2.8 | 107 | 107 ± 4.4 | 104.3 | 104 ± 3.2 | 106 | 106 ± 3.5 |
| | 500 | 498 | 99.6 ± 3.5 | 509 | 102 ± 1.7 | 487 | 97.5 ± 3.2 | 498 | 100 ± 3.3 |

Cnominal, theoretical concentration; CO, average concentration of seven samples; CV %, Coefficient of variance; 4-HIAA. 4-hydroxy-indole-3-acetic acid.

Overall, the analytical method was reliable to analyse both analytes in human plasma samples.

Selectivity and Sensitivity

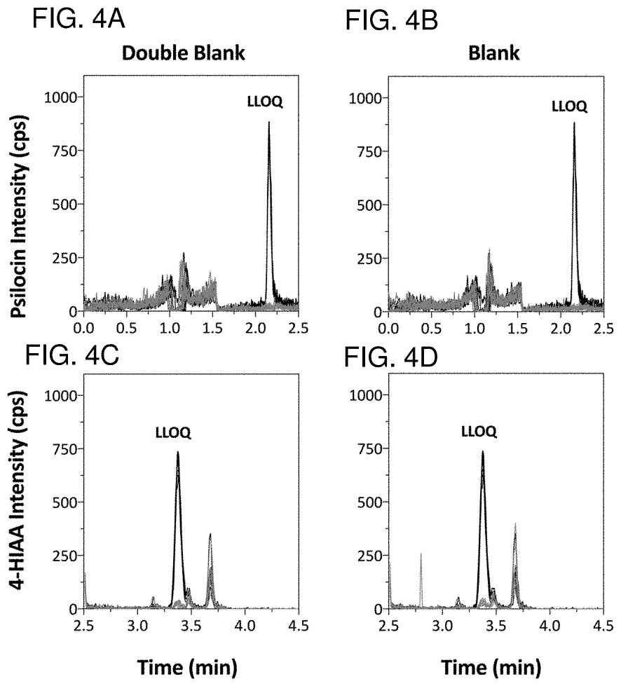

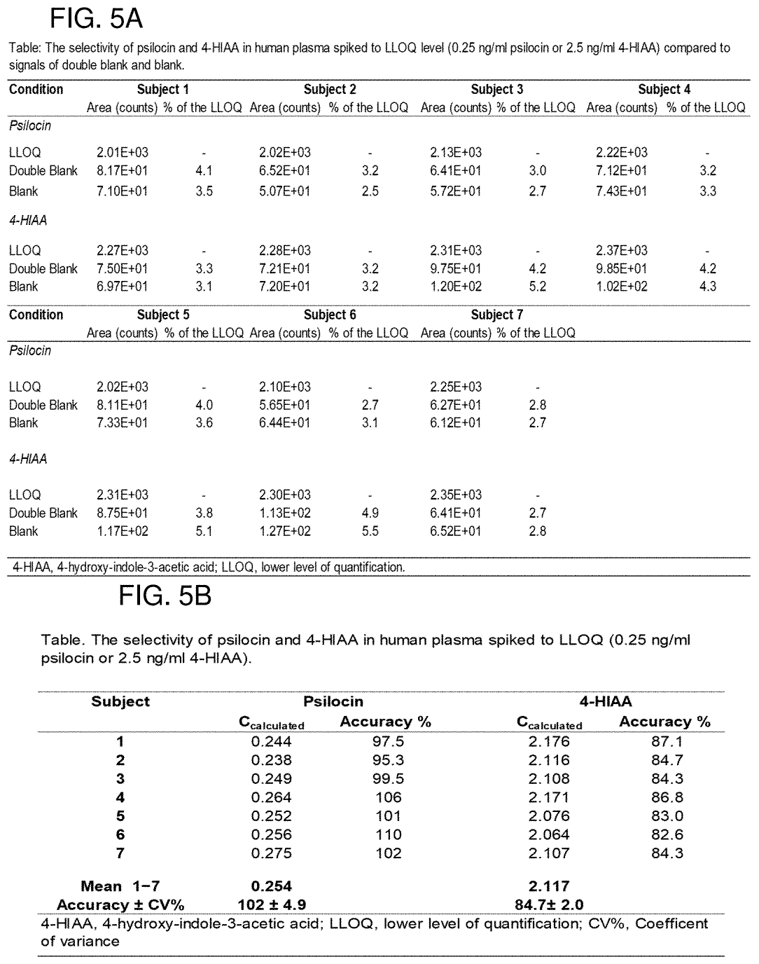

The selectivity and sensitivity of psilocin and 4-HIAA were assessed by comparing the LLOQ signal intensity of seven different batches of plasma to the respective baseline signal in blank and double blank samples. As shown in FIGS. 4A-4D, endogenous plasma components did not interfere with detection of psilocin and 4-HIAA. In FIGS. 4A-4D, seven double blank, blank and lower limit of quantification (LLOQ) samples (psilocin: 0.25 ng/mL, 4-HIAA: 2.5 ng/ml) were prepared using different batches of plasma. The LLOQ chromatograms (black lines) were overlaid with double blank (left) and blank (right) chromatograms (grey lines). FIGS. 4A and 4B correspond to psilocin while FIGS. 4C and 4D relate to 4-HIAA. The background noise determined in double blank samples did not interfere with the detection of psilocin or 4-HIAA as it accounted only for ≤4.1% and ≤5.5%, respectively of the observed LLOQ peak area. In addition, the internal standards, psilocin-d₁₀ and tryptophan-d₅, did not affect the selectivity of the analysis regarding the baseline noise recorded for blank samples. More precisely, psilocin and 4-HIAA background noise accounted for ≤4.1% and ≤5.5% of the LLOQ peak area, respectively (FIGS. 5A and 5B).

Blank plasma samples of seven different donors were spiked at the LLOQ level to evaluate if the analytes can be reliably quantified irrespective of the employed plasma source. A mean accuracy of 102% (95.3 to 110%) and 84.7% (82.6 to 87.1%) was determined for psilocin and 4-HIAA, respectively. None of the LLOQ samples were outside 80-120% accuracy and the precision was ≤1.9% for all seven batches of plasma.

These findings show that the method is selective for the quantification of psilocin and 4-HIAA in human plasma and that the plasma matrix does not affect the sensitivity of the analysis.

Recovery and Matrix Effect

The recovery and matrix effect of psilocin and 4-HIAA were examined after deproteinization of seven different plasma batches including four QC concentration levels:

TABLE 3
The recovery and matrix effect of psilocin
and 4-HIAA determined in human plasma.

| | Cₙₒₘᵢₙₐₗ | RRE ± CV | Mean ± CV | ME ± CV | Mean ± CV |
|---|---|---|---|---|---|
| Analyte | (ng/ml) | (%) | (%) | (%) | (%) |
| Psilocin | 0.25 | 94.1 ± 5.9 | 96.5 ± 2.2 | 129 ± 7.8 | 114 ± 13 |
| | 0.5 | 95.9 ± 4.5 | | 96.2 ± 7.4 | |
| | 10 | 99.2 ± 5.4 | | 106 ± 5.8 | |
| | 50 | 96.6 ± 7.2 | | 122 ± 5.9 | |
| 4-HIAA | 2.5 | 89.5 ± 8.2 | 94.7 ± 4.1 | 78.6 ± 3.0 | 69.5 ± 7.8 |
| | 5.0 | 95.0 ± 9.9 | | 62.7 ± 6.3 | |
| | 100 | 98.9 ± 9.3 | | 67.3 ± 6.1 | |
| | 500 | 95.3 ± 10 | | 69.8 ± 6.8 | |

Cₙₒₘᵢₙₐₗ, theoretical concentration; RRE, relative recovery; ME, matrix effect; CV %, Coefficent of variance; 4-HIAA, 4-hydroxy-indole-3-acetic acid.

The protein precipitation extraction was almost complete yielding a mean recovery of 96.5% for psilocin and 94.7% for 4-HIAA. Importantly, the bias between different plasma batches was smaller than 10.1% and consistent over all QC levels (CV %: ≤4.1%).

The psilocin signal in plasma extracts was on average 14% larger than in pure water. The signal intensity was even smaller when psilocin was solved in a water methanol mixture (1:4 v/v), mainly because the peaks were generally wider with an obvious peak fronting. Thus, the plasma matrix rather improved the binding of psilocin on the analytical column than increasing the ionisation efficiency in the mass spectrometer. In contrast, the 4-HIAA signal was suppressed by the plasma matrix by approximately 30%. Interestingly, the methanol content in the matrix-free sample did not affect the 4-HIAA peak shape. The seven plasma batches resulted in very similar matrix effect (CV %: ≤7.8%), which were independent from the utilized analyte concentration (CV %: ≤13%).

In summary, the employed extraction method recovered almost all psilocin and 4-HIAA from plasma and resulted in consistent and negligible matrix effects.

Stability

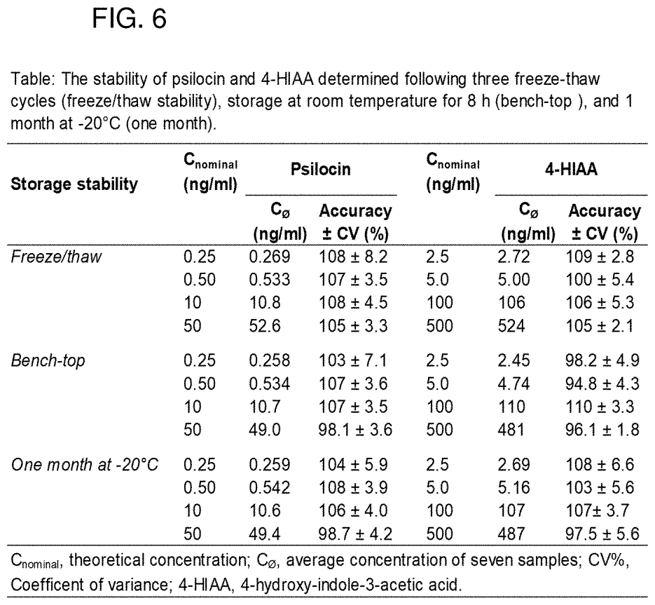

The stability of psilocin and 4-HIAA were examined after three freeze and thaw cycles as well as after 8 hours storage at room temperature and one month at −20° C. (FIG. 6).

Three repetitive freeze and thaw cycles did not decrease the stability of the analytes, because the accuracy of the QC samples was between 105-108% (CV %: ≤8.2%) for psilocin and between 100-109% (CV %: ≤5.4%) for 4-HIAA. Moreover, plasma samples that were stored for eight hours at room temperature or for one month at −20° C. contained similar amounts of psilocin and 4-HIAA in comparison to fresh samples (accuracy: 94.8-110%, CV %: ≤7.1%).

These results indicate that psilocin and 4-HIAA are stable under various conditions encountered in a laboratory, in support of previous studies evaluating the short term and freeze/thaw stability of psilocin (Martin et al., 2012).

Clinical Application

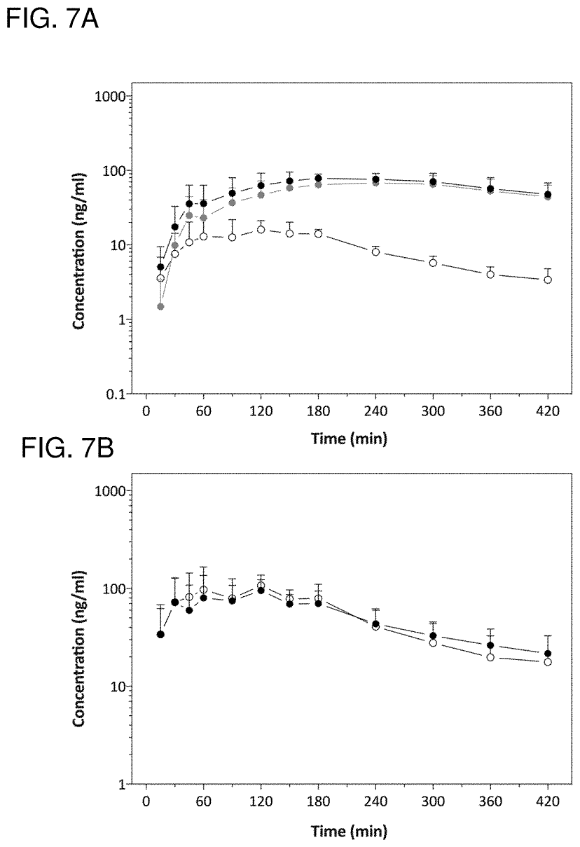

The application of the method was assessed by analysing the PK of psilocin and 4-HIAA in three healthy volunteers treated with an oral dose of 25 mg psilocybin (FIGS. 7A-7B and TABLE 4). An oral dose of 25 mg psilocybin was administered to three healthy volunteers. Plasma concentrations of psilocin and 4-HIAA were quantified before and up to seven hours post-treatment. All samples were reanalysed after deglucuronidation with Escherichia coli glucuronidase. FIG. 7A shows the concentration-time profile of psilocin, while FIG. 7B depicts the profile of 4-HIAA. White symbols correspond to unconjugated psilocin and 4-HIAA. The total amount of the conjugated and unconjugated metabolites is illustrated in black. The grey symbols show the difference between samples that were incubated with and without glucuronidase corresponding to the total amount of conjugated metabolites. A large proportion of psilocin underwent glucuronidation, whereas 4-HIAA was not conjugated. Mean values and the standard error of the mean are illustrated.

TABLE 4
The pharmacokinetic parameters of psilocin, psilocin-glucuronide,
and 4-HIAA found in plasma from three healthy volunteers
treated with an oral dose of 25 mg psilocybin.

| | Cₘₐₓ ± | Tₘₐₓ ± | AUCₗₐₛₜ | t_{1/2} |
|---|---|---|---|---|
| Analyte | SD (min) | SD (min) | (ng*min*ml⁻¹) | (min) |
| Psilocin | 19.2 ± 4.0 | 140 ± 46 | 3670 ± 780 | 127 ± 18 |
| Psilocin-glucuronide | 78.3 ± 7.9 | 220 ± 92 | 20631 ± 552 | 215 ± 72 |
| 4-HIAA | 137 ± 22 | 120 ± 60 | 22330 ± 992 | 139 ± 63 |

4-HIAA, 4-hydroxy-indole-3-acetic acid; Cₘₐₓ, maximal plasma concentration; Tₘₐₓ, time to reach maximal plasma concentration; AUCₗₐₛₜ, area under the plasma concentration time curve until 7 h post treatment; t_{1/2}, drug half life.

The maximal plasma level of psilocin and 4-HIAA was on average 19.2 ng/ml (SD: 4.0 ng/ml) and 137.3 ng/ml (SD: 22.0 ng/ml), respectively. Psilocin and 4-HIAA reached Tₘₐₓ approximately after 120-140 minutes post-treatment. The t_{1/2} of psilocin and 4-HIAA were estimated to be 127 minutes (SD: 18 minutes) and 139 minutes (SD: 63 minutes), respectively. Overall, the amount of 4-HIAA as depicted by the AUC_{LAST} was about 5 times larger than that of psilocin, which is in line with observations made by (Hasler et al., 1997).

In contrast to 4-HIAA, psilocin underwent extensive 0-glucuronidation. The psilocin-glucuronide reached on average a Cₘₐₓ of 78.3 ng/ml (SD: 7.9 ng/ml) after roughly 220 minutes. The calculated AUC_{LAST} of psilocin-glucuronide was 20631 ng·min·ml⁻¹ (SD: 552 ng·min·ml⁻¹) and thus 5-6 fold higher compared to the AUC_{LAST} of psilocin. This result agrees with previous studies, which reported that the majority of psilocin is conjugated by glucuronidation (Grieshaber et al., 2001; Kamata et al., 2006; Sticht & Kaferstein, 2000).

Importantly, the accuracy of the QC samples was between 93.6-113% and the precision ≤8.1% showing that the analytical run passed the acceptance criteria. Moreover, psilocin and 4-HIAA could always be quantified within the sampling period, as the observed concentrations were between 0.36 to 94.1 ng/ml for psilocin and 7.2 to 156.7 ng/ml for 4-HIAA. The herein presented method is therefore suitable for quantification of the clinical samples.

### CONCLUSION

Compared to other bioanalytical methods that measure psilocybin in human plasma, the currently invented method required only small amounts of sample and featured a straightforward extraction procedure, which facilitated an efficient analysis. The extraction protocol resulted in an almost complete analyte recovery. Consistent matrix effects were observed among various plasma batches, moreover the matrix did not interfere with the analysis of psilocin or 4-HIAA. The quantification of both analytes was accurate and precise within the chosen calibration range and compatible with observed levels in humans dosed with psilocybin. Overall, the current bioanalytical method is an important tool to further progress the development of psilocybin as a therapeutic agent.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

1. Björnstad K, Hultén P, Beck O, & Helander A (2009). Bioanalytical and clinical evaluation of 103 suspected cases of intoxications with psychoactive plant materials. Clinical toxicology 47: 566-572.
2. Bogenschutz M P, Podrebarac S K, Duane J H, Amegadzie S S, Malone T C, Owens L T, Ross S, & Mennenga S E (2018). Clinical Interpretations of Patient Experience in a Trial of Psilocybin-Assisted Psychotherapy for Alcohol Use Disorder. Front Pharmacol 9: 100.
3. Brown R T, Nicholas C R, Cozzi N V, Gassman M C, Cooper K M, Muller D, Thomas C D, Hetzel S J, Henriquez K M, Ribaudo A S, & Hutson P R (2017). Pharmacokinetics of escalating doses of oral psilocybin in healthy adults. Clin Pharmacokinet 56: 1543-1554.
4. Carhart-Harris R L, Roseman L, Bolstridge M, Demetriou L, Pannekoek J N, Wall M B, Tanner M, Kaelen M, McGonigle J, Murphy K, Leech R, Curran H V, & Nutt D J (2017). Psilocybin for treatment-resistant depression: fMRI-measured brain mechanisms. Sci Rep 7: 13187.
5. del Mar Ramirez Fernandez M, Laloup M, Wood M, De Boeck G, Lopez-Rivadulla M, Wallemacq P, & Samyn N (2007). Liquid chromatography-tandem mass spectrometry method for the simultaneous analysis of multiple hallucinogens, chlorpheniramine, ketamine, ritalinic acid, and metabolites, in urine. J Anal Toxicol 31: 497-504.
6. European Medicines Agency EMA (2011). Guideline on Bioanalytical Method Validationed. Agency E.M.: London, United Kingdom.
7. Grieshaber A F, Moore K A, & Levine B (2001). The detection of psilocin in human urine. J Forensic Sci 46: 627-630.
8. Griffiths R R, Johnson M W, Carducci M A, Umbricht A, Richards W A, Richards B D, Cosimano M P, & Klinedinst M A (2016). Psilocybin produces substantial and sustained decreases in depression and anxiety in patients with life-threatening cancer: a randomized double-blind trial. J Psychopharmacol 30: 1181-1197.
9. Grob C S, Danforth A L, Chopra G S, Hagerty M, McKay C R, Halberstadt A L, & Greer G R (2011). Pilot study of psilocybin treatment for anxiety in patients with advanced-stage cancer. Arch Gen Psychiatry 68: 71-78.
10. Hasler F, Bourquin D, Brenneisen R, Bar T, & Vollenweider F X (1997). Determination of psilocin and 4-hydroxyindole-3-acetic acid in plasma by HPLC-ECD and pharmacokinetic profiles of oral and intravenous psilocybin in man. Pharm Acta Helv 72: 175-184.
11. Hasler F, Bourquin D, Brenneisen R, & Vollenweider F X (2002). Renal excretion profiles of psilocin following oral administration of psilocybin: a controlled study in man. J Pharm Biomed Anal 30: 331-339.
12. Hofmann A, Heim R, Brack A, Kobel H, Frey A, Ott H, Petrzilka T, & Troxler F (1959). Psilocybin and Psilocin, zwei psychotrope Wirkstoffe aus mexikanischen Rauschpilzen. Helvetica Chimica Acta 42: 1557-1572.
13. Johnson M W, Garcia-Romeu A, & Griffiths R R (2017). Long-term follow-up of psilocybin-facilitated smoking cessation. Am J Drug Alcohol Abuse 43: 55-60.
14. Kamata T, Nishikawa M, Katagi M, & Tsuchihashi H (2003). Optimized glucuronide hydrolysis for the detection of psilocin in human urine samples. Journal of Chromatography B 796: 421-427.
15. Kamata T, Nishikawa M, Katagi M, & Tsuchihashi H (2006). Direct detection of serum psilocin glucuronide by LC/MS and LC/MS/MS: time-courses of total and free (unconjugated) psilocin concentrations in serum specimens of a “magic mushroom” user. Forensic Toxicology 24: 36-40.
16. Kolaczynska K E, Liechti M E, & Duthaler U (2021). Development and validation of an LC-MS/MS method for the bioanalysis of psilocybin's main metabolites, psilocin and 4-hydroxyindole-3-acetic acid, in human plasma. J Chromatogr B Analyt Technol Biomed Life Sci 1164: 122486.
17. Lindenblatt H, Kramer E, Holzmann-Erens P, Gouzoulis-Mayfrank E, & Kovar K-A (1998). Quantitation of psilocin in human plasma by high-performance liquid chromatography and electrochemical detection: comparison of liquid-liquid extraction with automated on-line solid-phase extraction. Journal of Chromatography B: Biomedical Sciences and Applications 709: 255-263.
18. Manevski N, Kurkela M, Hoglund C, Mauriala T, Court M H, Yli-Kauhaluoma J, & Finel M (2010). Glucuronidation of psilocin and 4-hydroxyindole by the human UDP-glucuronosyltransferases. Drug Metab Dispos 38: 386-395.
19. Martin R, Schurenkamp J, Pfeiffer H, & Köhler H (2012). A validated method for quantitation of psilocin in plasma by LC-MS/MS and study of stability. International journal of legal medicine 126: 845-849.
20. Moreno F A, Wiegand C B, Taitano E K, & Delgado P L (2006). Safety, tolerability, and efficacy of psilocybin in 9 patients with obsessive-compulsive disorder. J Clin Psychiatry 67: 1735-1740.
21. Nichols D E (2004). Hallucinogens. Pharmacol Ther 101: 131-181.
22. Passie T, Seifert J, Schneider U, & Emrich H M (2002). The pharmacology of psilocybin. Addict Biol 7: 357-364.
23. Rickli A, Moning O D, Hoener M C, & Liechti M E (2016). Receptor interaction profiles of novel psychoactive tryptamines compared with classic hallucinogens. Eur Neuropsychopharmacol 26: 1327-1337.
24. Ross S, Bossis A, Guss J, Agin-Liebes G, Malone T, Cohen B, Mennenga S E, Belser A, Kalliontzi K, Babb J, Su Z, Corby P, & Schmidt B L (2016). Rapid and sustained symptom reduction following psilocybin treatment for anxiety and depression in patients with life-threatening cancer: a randomized controlled trial. J Psychopharmacol 30: 1165-1180.
25. Sewell R A, Halpern J H, & Pope H G, Jr. (2006). Response of cluster headache to psilocybin and LSD. Neurology 66: 1920-1922.
26. Sticht G, & Kaferstein H (2000). Detection of psilocin in body fluids. Forensic Sci Int 113: 403-407.
27. Vollenweider F X, Vollenweider-Scherpenhuyzen M F, Babler A, Vogel H, & Hell D (1998). Psilocybin induces schizophrenia-like psychosis in humans via a serotonin-2 agonist action. Neuroreport 9: 3897-3902.

What is claimed is:

1. A method of measuring and identifying metabolites of a tryptamine compound, including the steps of:
obtaining a sample from an individual; and
measuring and identifying metabolites of the tryptamine compound in the sample by performing a LC-MS/MS analysis.

2. The method of claim 1, wherein the sample is chosen from the group consisting of plasma, blood, urine, and saliva.

3. The method of claim 1, wherein the tryptamine compound is chosen from the group consisting of psilocybin, psilocybin prodrugs, psilocybin analogs, dimethyltryptamine (DMT), DMT analogs, and DMT prodrugs.

4. The method of claim 1, wherein the tryptamine compound is psilocybin and wherein the metabolite is chosen from the group consisting of psilocin, 4-hydroxyindole-3-acetic acid (4-HIAA), psilocin-O-glucuronide, 4-hydroxytryptophol (4-HTP), and combinations thereof.

5. The method of claim 1, further including the step of determining that an individual has taken a tryptamine compound.

6. The method of claim 1, further including the step of determining if the tryptamine compound is metabolized effectively by the individual.

7. The method of claim 1, wherein the tryptamine compound is psilocybin and further including the step of analysing a ratio of psilocin to metabolites.

8. The method of claim 1, wherein said performing a LC-MS/MS analysis further includes the steps of separating analytes using a modular ultra-high performance liquid chromatography system, performing electrospray ionisation, and detecting analytes by multiple reaction monitoring.

9. The method of claim 1, wherein said sample is obtained at a time chosen from the group consisting of 0, 15, 30, 45, 60, 90, 120, 150, 180, 240, 300, 360, and 420 minutes after treatment of the individual with the tryptamine compound.

10. A method of adjusting dosing in patients with tryptamine compound-assisted psychotherapy in therapeutic drug monitoring (TDM), including the steps of:
obtaining a sample from an individual;
measuring and identifying metabolites of the tryptamine compounds in the sample by performing a LC-MS/MS analysis; and
adjusting a dose of the tryptamine compounds based on the measured metabolites.

11. The method of claim 10, wherein the sample is chosen from the group consisting of plasma, blood, urine, and saliva.

12. The method of claim 10, wherein the tryptamine compound is chosen from the group consisting of psilocybin, psilocybin prodrugs, psilocybin analogs, dimethyltryptamine (DMT), DMT analogs, and DMT prodrugs.

13. The method of claim 10, wherein the tryptamine compound is psilocybin and wherein the metabolite is chosen from the group consisting of psilocin, 4-hydroxyindole-3-acetic acid (4-HIAA), psilocin-O-glucuronide, 4-hydroxytryptophol (4-HTP), and combinations thereof.

14. The method of claim 10, further including the step of determining if the tryptamine compound is metabolized effectively by the individual.

15. The method of claim 14, wherein the tryptamine compound is psilocybin and further including the step of analysing a ratio of psilocin to metabolites.

16. The method of claim 10, wherein said performing a LC-MS/MS analysis further includes the steps of separating analytes using a modular ultra-high performance liquid chromatography system, performing electrospray ionisation, and detecting analytes by multiple reaction monitoring.

17. The method of claim 10, wherein said sample is obtained at a time chosen from the group consisting of 0, 15, 30, 45, 60, 90, 120, 150, 180, 240, 300, 360, and 420 minutes after treatment of the individual with the tryptamine compound.
